(19) United States
(12) Patent Application Publication
(10) Pub. No.: US 2004/0115289 A1
(43) Pub. Date: Jun. 17, 2004

(54) Cosmetic or medicinal composition containing sesquiterpene lactone or the like for treating hair -growth related disorders, and preparation method

(76) Inventors: Daniel Jean, Vic-le-Comte (FR); L?eacute;on Cariel, Paris (FR)

Correspondence Address: D Douglas Price, Steptoe & Johnson, 1330 Connecticut Avenue N W, Washington, DC, 20036, US

(21) Appl. No.: 10/204,018

(22) PCT Filed: Feb. 16, 2001
(86) PCT No.: PCT/FR01/00471

(30) Foreign Application Priority Data
Feb. 18, 2000 (FR) 00/02023

(51) Int. Cl. A61K 35/78; A61K 31/366

(52) U.S. Cl. 424/764; 514/460

(57) ABSTRACT

The invention concerns a cosmetic or medicinal composition for topical application. The invention is characterised in that said composition comprises as active principle at least a sesquiterpene lactone, such as for example helenalin, dihydrohelenalin, parthenolide, and cnicin, or a sesquiterpene lactone analogue in a physiologically acceptable medium and suitable for topical application.

The present invention relates to a composition, more particularly a cosmetic composition, intended for the treatment of alopecia and related conditions, to the use of a composition in the manufacture of a medicament for treating these disorders, to a process for the preparation of the said compositions and to a cosmetic treatment process.

Hair loss can be brought about by various causes, such as age, a pathology (pityriasis capitis) or external attack (bleaching or dyeing). It can be local or generalized and can cause annoyance simply of an aesthetic nature or can constitute a true pathology.

A number of products have been provided in the past for stimulating hair growth, both in men and in women, but none to date has produced a clearcut result in a significant proportion of the population subjected to the treatment.

Irritant products (capsaicin) or vasodilators (pilocarpine), and also more recently antihypertensive molecules (minoxidil and derivatives), have been provided. The latter products have proved to bring about hypertrichosis in patients treated for hypertension by the systemic route and to stimulate hair growth by the topical route in patients exhibiting the beginning of or longer established alopecia.

However, these effects have been shown to be positive for a minority of patients and to be relatively subdued in all cases.

In the same way as these products, marketed in cosmetics or pharmacology for their real or supposed effects on the hair, mention may also be made of the case of immunosuppressant molecules, cyclosporin and FK506 (fungal macrolide), which have a similar effect.

Cyclosporin is active by the systemic route or by the local route, whereas FK506 is active only by the local route.

These products also induce in a clearcut way the growth of hairs in the C57/B16 black mouse, which is the reference animal for screening studies for molecules which are active with respect to hair growth. This induction of growth is displayed by a transition from the telogenic phase of the hairs to the anagenic phase which is easy to observe and to measure by virtue of the pigmentation of the hairs, whereas the skin of the mouse in the telogenic phase is not pigmented or only slightly pigmented.

However, the immunosuppressant effects of cyclosporin and FK506 do not allow them to be used in cosmetics or even in aesthetic medicine. As suppressing the local immune system can induce serious disorders at the cutaneous level, such as cancers or opportunistic infections, these products can hardly be used in these fields.

Consequently, it would be very useful to have available products capable of exhibiting a clearcut effect with regard to hair growth in order to treat in particular androgenic alopecia, which is one of the most important aesthetic problems for man.

This is why the present invention relates to a composition intended for a topical application, in particular a cosmetic application, characterized in that it comprises, as active principle, at least one sesquiterpene lactone or one analogue of the sesquiterpene lactones in a physiologically acceptable medium appropriate for a topical application, the said composition being more particularly intended to treat alopecia, to stimulate pilar growth, to stimulate capillary growth and/or to treat seborrhoeic conditions and related conditions.

The term “physiologically acceptable medium appropriate for a topical application” is understood to denote a medium which is compatible with the active principle and which is acceptable in cosmetics or in pharmaceuticals, in particular which is compatible with the skin and the scalp.

The sesquiterpene lactones which can be used in the compositions according to the present invention are described in particular in “Pharmacognosie, Phytochimie et Plantes Medicinales” [Pharmacognosy, Phytochemistry and Medicinal Plants] by Bruneton J., published by Lavoisier, Paris, 2nd edition, 1993, pages 499-510.

The term “analogues of sesquiterpene lactones”, within the meaning of the present invention, is understood to mean the derived or substituted forms of these lactones having an alpha-methylene-gamma-lactone group and their bioequivalent derivatives defined by the compounds exhibiting an active structure having properties with respect to pilar growth (that is to say, promoting the passage of the hair from the anagenic phase to the telogenic phase) which are similar to those demonstrated in the present text for helenalin, dehydrohelenation, parthenolide and cnicin.

According to a specific embodiment of the present invention, the sesquiterpene lactone is preferably chosen from the group consisting of helenalin, dihydrohelenalin, parthenolide, cnicin and their derivatives. The term “derivatives” is understood to mean essentially the esters, in particular the fatty acid esters, of these lactones.

The sesquiterpene lactones are present in particular in plant extracts, in particular in the Asteracae and especially in extracts of Arnica montana, of Tanacetum parthenium and of Cnicus benedictus. This is why the invention also relates to a composition intended for a topical application, in particular a cosmetic application, comprising, as active principle, a plant extract rich in sesquiterpene lactone, in particular an extract of a plant of the plant species of the family of the Asteracae, for example Arnica montana, Tanacetum parthenium and Cnicus benedictus. The plant extract is preferably an extract of the flower for Arnica montana and the extract of the flowering aerial parts for Tanacetum parthenium and Cnicus benedictus.

These extracts, obtained from various plant species of the family of the Asteracae, all comprise compounds belonging to the chemical family of the sesquiterpene lactones.

In an entirely surprising way, these substances of plant origin proved to have a very marked effect on the induction of the anagenic phase of the hair in the rabbit or in the C57/B16 mouse.

These lactones are known to have various pharmacological properties related to the anti-inflammatory or antitumour effect, but it has never been indicated that these molecules could have the slightest effectiveness on the hair cycles or pilar growth. Without wishing to be bound to any theory, the biological effectiveness generally of these substances is to be related to the presence of certain functional groups within the molecule and in particular the alpha-methylene-gamma-lactone group.

Although the active principles according to the present invention can be obtained by chemical synthesis or by semisynthesis, use will preferably be made of plant extracts rich in sesquiterpene lactones.

This is why the present invention also relates to processes for the preparation of plant extracts rich in sesquiterpene lactones, in particular Asteracae extracts, obtained by a process comprising the following stages:

a) extraction of at least a portion of the plant, in particular of the flowers and of the aerial parts of plants of the family of the Asteracae, by means of an aqueous/organic solution comprising a water-miscible organic solvent, followed by evaporation of the said solvent, and

b) extraction of the resulting aqueous phase by means of a water-immiscible organic solvent, then

c) recovery of the extract present in the said organic phase.

In the implementation of the process described above, the part of the plant extracted will preferably be the flower or the aerial parts richer in sesquiterpene lactones. The water-miscible organic solvent used for the first decoctions is chosen from alcohols comprising from 1 to 5 carbon atoms, acetonitrile, tetrahydrofuran and acetone. This solvent is preferably an alcohol of low molecular weight, such as methanol, ethanol or an alcohol comprising from 3 to 5 carbon atoms. More preferably still, ethanol will be used. The solution comprising water and a water-miscible organic solvent preferably comprises from 40 to 60% by weight of water. The stage a) can comprise several extractions.

The extraction of the aqueous phase obtained is preferably carried out with a water-immiscible organic solvent chosen from ethers, such as ethyl ether. Other organic solvents can nevertheless be used, such as, for example, methylene chloride, chloroform and ethyl acetate.

An organic phase comprising the extract rich in sesquiterpene lactones is then obtained, which phase will optionally be purified, in particular by chromatography and, for example, by redissolving and chromatographing the solution.

Chromatography can in particular be carried out by using, as support, a silica gel, after attaching the active principle in a medium of alkane type, such as hexane, and then eluting using a chlorinated solvent, such as chloroform, for example.

It is possible to carry out an additional or alternative purification stage using preparative chromatography carried out, for example, using, as support, a liphophilic gel (Sephadex LH20), in a medium of aqueous/organic solution type, such as 50% methanol, and then eluting with this same aqueous/organic solution or another aqueous/organic solution, such as 70% ethanol, or a pure organic solution, such as acetone.

Other purification methods can, of course, be used to purify the extracts rich in sesquiterpene lactones.

The topical compositions, in particular cosmetic compositions, according to the present invention can, of course, be provided in the forms which are usually known for this type of administration, that is to say in particular lotions, foams, gels, dispersions, sprays or creams, for example, but can also be administered in shampoos, with excipients which make possible in particular cutaneous penetration, in order to improve the properties and the accessibility of the active principle.

These compositions, essentially intended for a topical application, comprise, in addition to the active principle, for example a plant extract, a physiologically acceptable medium, generally based on water or on solvent, for example alcohols, ethers or glycols.

The compositions according to the present invention preferably comprise from 0.001 to 10% by weight of sesquiterpene lactone.

These compositions can also comprise:

surface-active agents,

preservatives,

stabilizing agents,

emulsifiers and

thickeners.

These components are known in the cosmetics field, in particular for the preparation of compositions for the treatment of hair loss.

The present invention additionally relates to a cosmetic treatment process such that a cosmetic composition, comprising at least one sesquiterpene lactone according to the invention and preferably comprising an extract rich in sesquiterpene lactones obtained from a plant of the family of the Asteracae, is applied topically, in particular to the scalp, in order to improve the appearance of the hair and/or to treat alopecia.

The present invention also relates to compositions comprising sesquiterpene lactones and the extracts described above in an application of pharmaceutical type. Particularly relevant will be cases where the alopecia or the seborrhoeic conditions have a pathological nature.

Thus, the present invention also relates to the use of a composition comprising at least one sesquiterpene lactone or one analogue of the sesquiterpene lactones in the manufacture of a medicament intended to treat alopecia or seborrhoeic conditions or to stimulate pilar growth and capillary growth or [lacuna] related conditions when the latter have a pathological nature.

The compositions comprising at least one sesquiterpene lactone mentioned above can, of course, be obtained as was described in the case of the cosmetic applications.

As was said above, preference will be given to sesquiterpene lactones chosen from the group consisting of helenalin, dihydrohelenalin, parthenolide, cnicin and their derivatives. Mention should more particularly be made, among these derivatives, of the esters and in particular the fatty acid esters of these lactones.

The medicaments comprising these active principles can be provided in the forms appropriate for their administration, that is to say in particular lotions, creams or sprays, or optionally, if this is necessary, can be presented in forms of injectable form type, in particular intradermal type.

Finally, the present invention relates to extracts of plants of the family of the Asteracae which are rich in sesquiterpene lactones such as can be obtained by the process described above.

Other characteristics and advantages of the present invention will result from the reading of the examples below.

### EXAMPLE 1

One kilo of dry flowers of Arnica montana are extracted by 4 decoctions at reflux for 30 minutes in 5 litres of ethanol comprising 50% water. The extractive solutions are combined and the alcohol is evaporated.

The aqueous phase is concentrated to one litre and extracted countercurrentwise with ethyl ether until the ether no longer has a significant residue on evaporation. The ethereal phase is dried over anhydrous sodium sulphate and evaporated.

9 g of residue (product 1) are obtained.

### EXAMPLE 2

Ten grams of product 1 are dissolved/suspended in 10 ml of hexane, and 1 g of silica gel 60 for column chromatography is added to a round-bottomed flask. The hexane is evaporated to dryness while continually stirring in order to ensure perfect homogenization of the combined mixture.

A chromatography column with a diameter of 4 cm and a height of 20 cm is then prepared with silica gel 60 in hexane. The product 1-chloroform gel mixture is placed at the surface of the gel. The fraction with is eluted with chloroform is collected. The chloroform is evaporated.

A residue of 0.19 g is obtained (product 2).

### EXAMPLE 3

Preparative chromatography is carried out on the product 2 on a silica layer with the following solvent: toluene/ethyl acetate 70/30 (v/v).

The portion of support between Rf 0.55 and the Rf 0.75, where greenish-yellow spots can be observed, is removed. The silica thus obtained is extracted 4 times at reflux with 50 ml of pure methanol. The methanol is evaporated to dryness.

40 mg of residue are obtained (product 3).

Analyses of the Products

The three products are analysed by gas chromatography coupled to a mass spectrometer.

| | |
|---|---|
| Column: | HP5 MS 5% phenylmethylsiloxane |
| Carrier gas: | helium, constant flow rate of |
| | 1 ml/min |
| Temperature programme: | 150° C. for 5 min and then |
| | increase by 5° C./min to 250° C. |
| Detection: | Total ion current between ion |
| | mass 50 and 500. |

The spectra relating to the various components show the predominant presence of sesquiterpene lactones: helenalin, dihydrohelenalin and their derivatives. According to the studies described in the bibliography, they are probably esters of fatty acids and of these lactones.

By this chromatography method, it can be shown that the products 1, 2 and 3 respectively comprise 17.5%, 42.6% and 43% by mass of helenalin+dihydrohelenalin with respect to the total mass of the composition.

The purification introduced between the products 2 and 3 consists especially of the removal of the chlorophyll pigments (decoloration).

### Pharmacological Study

1. Test on the Rabbit

Male rabbits aged at least 12 weeks and identical in age, in order for the hairs of these animals to be in the telogenic phase, which can be confirmed by shearing the animals, are used.

The test products are dissolved in pure ethanol in the proportion of a concentration of 25% (w/v). This solution is then diluted 1/10 with sweet almond oil. A concentration of active product of 2.5% is then obtained.

0.2 ml of these solutions are deposited on a gauze applied to the skin and a control, obtained by preparing the same solvent as that which was used to dissolve the active principles, is also deposited. The products and the control are left in contact with the skin for 15 minutes and then the gauze is removed. The treatment is carried out for 2 days at the rate of three applications per day at intervals of four hours.

The difference between the mean time taken by the hairs to grow on the treated parts and the control parts is subsequently evaluated.

For the three products used, a difference of more than 2 fold is found.

However, growth is faster for the products 2 and 3 than for the product 1.

As a marked enrichment in sesquiterpene lactones is found from the product 1 to the products 2 and 3, it may be concluded therefrom that these substances are responsible for the activity.

A solution comprising 1% of pure commercial parthenolide (Aldrich, ref. 38,428-3) and a solution comprising 1% of pure commercial cnicin (Extrasynthese, ref. 8725) are prepared in the same excipient as the Arnica montana extract and are subjected to the test on the rabbit.

A substantially equivalent growth of the hairs is found between the Arnica extract and the parthenolide and a growth approximately two times worse is found for the cnicin.

2. Tests on the C57/B16 Mouse

Female mice aged 5 weeks, all identical, are used. The mice are shown on the back, so as to render the hairs unnoticeable.

0.2 ml of one of each product in the same solvent as for the test on the rabbits and at the same concentration is deposited on the backs of the mice.

A test is thus carried out by comparison between different mice, in contrast to the test on the rabbit, where the same animal represents its own control.

The product is deposited on the back, without using gauze, once daily for 5 days in a row. It is found that, in the treated mice, the hairs grow very quickly. They become visible on the second day of treatment to reach approximately 1 mm from the third day, whereas the hairs begin to grow on the control mice only from the third week after the beginning of the treatment.

Just as for the rabbit, it is found that the effect is most marked for the products 2 and 3 than for the product 1.

A solution comprising 1% of pure commercial parthenolide (Aldrich, ref. 38,428-3) and a solution comprising 1% of pure commercial cnicin (Extrasynthese, ref. 8725) are prepared in the same excipient as the Arnica montana extract and are subjected to the test on the rabbit.

A substantially equivalent growth of the hairs is found between the 2.5% Arnica extract and the 1% parthenolide and a growth approximately two times worse is found for the 1% cnicin.

These results show that the compositions studied are effective in the treatment of pathologies related to pilar disorders.

### EXAMPLE 4

One kilo of flowering aerial parts of Tanacetum parthenium is extracted by four decoctions at reflux for 30 minutes in 5 litres of ethanol comprising 50% water.

The extractive solutions are combined and the alcohol is evaporated.

The aqueous phase is concentrated to 1 litre and extracted countercurrentwise with ethyl ether until the ether no longer has a significant residue on evaporation.

The ethereal phase is dried over anhydrous sodium sulphate and evaporated.

26 g of residue are obtained.

The ethereal phase is taken up in one litre of methanol comprising 50% water and passed through a column with a diameter of 5 cm comprising 100 g of Sephadex LH20 in suspension in 50% methanol.

Elution is carried out with three litres of this same solvent. The solvent is evaporated to dryness under reduced pressure.

10.5 g of residue are obtained.

This residue is subjected to analysis by gas chromagraphy coupled to a mass spectrometer:

| | |
|---|---|
| Column: | HP5 MS 5% phenylmethylsiloxane |
| Carrier gas: | helium, constant flow rate of |
| | 1 ml/min |
| Temperature programme: | 150° C. for 5 min and then |
| | increase by 5° C./min to 250° C. |
| Detection: | Total ion current between ion |
| | mass 50 and 500. |

The spectra relating to the various components show the predominant presence of sesquiterpene lactones: parthenolide, seco-tanapartholide A, canin, tanaparthin-alpha-peroxide and their derivatives.

Furthermore, quantification carried out with a parthenolide control of commercial origin (Aldrich, ref. 38,428-3) indicates that the final extract comprises 32% of parthenolide.

### EXAMPLE 5

One kilo of flowering aerial parts of Cnicus benedictus is extracted by 4 decoctions at reflux for 30 minutes in 5 litres of methanol comprising 60% water.

The extractive solutions are combined and the methanol is evaporated.

The aqueous phase is concentrated to 0.5 litre and extracted countercurrentwise with ethyl ether until the ether no longer has a significant residue on evaporation.

The ethereal phase is dried over anhydrous sodium sulphate and evaporated.

10.7 g of residue are obtained.

The ethereal phase is taken up in one litre of methanol comprising 50% water and passed through a column with a diameter of 5 cm comprising 100 g of Sephadex LH20 in suspension in 50% methanol.

Elution is carried out with three litres of the same solvent.

The solvent is evaporated to dryness under reduced pressure.

620 mg of residue are obtained.

This residue is subjected to analysis by gas chromatography coupled to a mass spectrometer:

| | |
|---|---|
| Column: | HP5 MS 5% phenylmethylsiloxane |
| Carrier gas: | helium, constant flow rate of |
| | 1 ml/min |
| Temperature programme: | 150° C. for 5 min and then |
| | increase by 5° C./min to 250° C. |
| Detection: | Total ion current between ion |
| | mass 50 and 500. |

The spectra relating to the various components show the predominant presence of sesquiterpene lactones, in particular cnicin and its derivatives.

Furthermore, quantification carried out with a cnicin control of commercial origin (Extrasynthese, ref. 8725) indicates that the final extract comprises 41% of cnicin.

1. Use of a composition comprising, as active principle, at least one sesquiterpene lactone or one analogue of the sesquiterpene lactones in the manufacture of a medicament or of a cosmetic composition intended to treat alopecia, to stimulate hair growth and/or to stimulate capillary growth.

2. Use according to claim 1, such that the sesquiterpene lactone is chosen from the group consisting of helenalin, dihydrohelenalin, parthenolide, cnicin and their derivatives.

3. Use according to either of claims 1 and 2, such that the lactone is obtained from an extract of a plant of the plant species of the family of the Asteracae, preferably an extract of Arnica montana, of Tanacetum parthenium or of Cnicus benedictus.

4. Use according to one of the preceding claims, such that the plant extract is an extract of the flower or of the aerial parts of the plant.

5. Use according to one of the preceding claims, such that the extract is obtained by the process comprising the following stages:
a) extraction of at least a portion of the plant, in particular the flowers and of the aerial parts of plants of the family of the Asteracae, by means of an aqueous/organic solution comprising a water-miscible organic solvent, followed by evaporation of the said solvent, and
b) extraction of the resulting aqueous phase by means of a water-immiscible organic solvent, then
c) recovery of the extract present in the said organic phase.

6. Use according to claim 5, such that the extract obtained in stage c) is purified by chromatography to produce a phase rich in sesquiterpene lactones.

7. Use according to one of the preceding claims, characterized in that the medicament is presented in the form of lotions, creams, sprays or forms of injectable form type.
